(19) United States
(12) Patent Application Publication
(10) Pub. No.: US 2006/0292173 A1
(43) Pub. Date: Dec. 28, 2006

(54) Multibacterial vaccines and uses thereof

(76) Inventor: Donald H. MaCadam, Port Dover (CA)

Correspondence Address: MCCARTHY TETRAULT LLP, BOX 48, SUITE 4700,, 66WELLINGTON STREET WEST, TORONTO, ON, M5K 1E6, CA

(21) Appl. No.: 11/450,313

(22) Filed: Jun. 12, 2006

Related U.S. Application Data
(63) Continuation-in-part of application No. 11/298,495, filed on Dec. 12, 2005.
(60) Provisional application No. 60/635,163, filed on Dec. 13, 2004.

(51) Int. Cl. A61K 39/116; A61K 31/739; C12Q 1/68; G01N 33/554; G01N 33/569

(52) U.S. Cl. 424/203.1; 435/6; 435/7.32; 514/54

(57) ABSTRACT

The present invention provides methods for establishing standards for Gram-negative, Gram-positive, and mixed bacterial cultures. The present invention also provides methods for reproducing Gram-negative, Gram-positive, and mixed bacterial cultures. The present invention further provides methods for preparing multibacterial vaccines. Also provided are multibacterial vaccines prepared in accordance with these methods, and methods for treating and/or preventing disorders using these multibacterial vaccines. In addition, the present invention provides methods for predicting the efficacy of multibacterial vaccines, and methods for enhancing the efficacy of multibacterial vaccines.

### RELATED APPLICATIONS

This application is a continuation-in-part of U.S. patent application Ser. No. 11/298,495, filed Dec. 12, 2005, which claims the benefit of U.S. Provisional Application No. 60/635,163, filed Dec. 13, 2004. The entire contents of the foregoing applications are incorporated herein by reference.

### FIELD OF THE INVENTION

The present invention generally relates to multibacterial vaccines, composed of whole-cell lysates of Gram-negative and Gram-positive bacteria, in which the relative concentrations of at least four immunostimulatory bacterial substances are known. More specifically, the present invention relates to Coley vaccines.

### BACKGROUND OF THE INVENTION

Live bacteria, bacterial whole-cell lysates, bacterial extracts, purified bacterial substances, and synthetic bacterial substances are used as pharmacological agents and in medical research. The live Bacillus Calmette-Guerin, an attenuated strain of Mycobacterium bovis, is a treatment of bladder carcinoma (PDR Nurses Drug Handbook, 2002); OK-432, a whole-cell lysate of Streptococcus pyogenes, is a non-small-cell lung cancer treatment (Sakamoto, 2001); various bacterial extracts have been used in the treatment of cancer (Nauts, 1984); the purified bacterial substance lipopolysaccharide (LPS) is widely used in immunological research; and synthetic analogues of bacterial DNA are being clinically tested in the treatment of cancer, hepatitis, asthma, and allergy (Coley Pharmaceuticals, 2003).

The use of a preparation of whole-cell lysates of Gram-negative and Gram-positive bacteria as a pharmacological agent dates from 1893, when Dr. William Coley developed a class of immunostimulatory vaccines known as “Coley Toxins”, “Coley's Mixed Fluid”, “Coley Vaccine”, or “Multi Bacterial Vaccine”(Coley, 1906; Wiemann, 1994). Multi Bacterial Vaccine has been used primarily in the treatment of cancer, but has also been used in the treatment of severe burns, infections, and radiation injury (Nauts, 1990; Waisbren, 1987).

In 1893, the first cancer patient to receive Multi Bacterial Vaccine was a sixteen-year-old boy with a massive abdominal tumour. Every few days, the vaccine was injected directly into the tumour mass. Upon each injection, there was a dramatic rise in body temperature, accompanied by extreme chills and trembling. The tumour gradually diminished in size. After four months of intensive treatment, the tumour was a fifth its original size; three months later, the remains of the growth were barely perceptible. The boy received no further anticancer treatment, and remained in good health until he died of a heart attack 26 years after receiving Multi Bacterial Vaccine therapy (Nauts, 1990).

A review of 897 cancer patients treated with Multi Bacterial Vaccine up to 121 years ago found that complete regression and 5-year survival occurred in 46% of the 523 inoperable cases and in 51% of the 374 operable cases (Nauts, 1982). These results are comparable to modem 5-year survival rates. The National Cancer Institute estimates overall 5-year cancer survival at 35% in 1950-54 and 63.8% in 1992-98 (SEER, 2003).

To determine comparable rates of 10-year survival, researchers compared 128 Multi Bacterial Vaccine patients treated in New York between 1890 to 1960 with 1,675 matched controls from the National Cancer Institute's Survival Epidemiology End Result database of patients diagnosed in 1983 and followed through 1993 (Richardson, 1999). The study found higher rates of 10-year survival for Multi Bacterial Vaccine patients as compared with modem patients in kidney cancer (33% to 23%), ovarian cancer (55% to 29%), and sarcoma (50% to 38%).

Between 1893-1959, at least 14 different formulations of Multi Bacterial Vaccines were administered to patients, and physicians reported significant variations in potency between the various formulations (Nauts, 1975). Some variations in potency—namely, those differences that could be overcome by titration of dose—were due to dilution factors. Other variations in potency could not be overcome by titration of dose, suggesting that the concentration of any one substance was less important than the relative concentrations of two or more substances.

The wide-ranging efficacies that are possible from a mixture of whole-cell lysates of Gram-negative and Gram-positive bacteria are demonstrated by the Havas experiments, in which different formulations of Multi Bacterial Vaccine were tested in mice with implanted tumours (Havas, 1958). In the Havas experiments, a Gram-positive bacterial culture was prepared by inoculating 0.1 mL of a 24-hour neopeptone broth culture (10 g neopeptone, 5 g NaCl, and 3 g beef extract per liter of double-distilled water) of Streptococcus pyogenes into 50 mL of neopeptone broth, incubating at 37° C., and growing for 4, 14, or 28 days. Four strains of Streptococcus pyogenes were used, and labelled ‘N’, ‘B’, ‘D’, and ‘E’. A Gram-negative culture was prepared by inoculating 0.1 mL of a 24-hour broth culture of Serratia marcescens into 50 mL of neopeptone broth, incubating at 25° C., and growing for 2, 7, or 14 days. One strain of Serratia marcescens was labelled ‘S’. The cultures were heat-sterilized at 68° C. for 90 minutes.

The cultures were either grown separately and mixed before heat sterilization, or grown together with Serratia marcescens inoculated into the already-growing Streptococcus pyogenes culture at the appropriate time. In the nomenclature, ‘s’ means grown separately and ‘t’ means grown together. For example, N14S7s includes Streptococcus pyogenes strain N grown for 14 days and Serratia marcescens grown separately for 7 days.

Swiss mice, 8-9 weeks old with Sarcoma 37 tumours ranging from 1.5 to 2.5 sq cm at the base, were used in the Havas experiments. The test dosage was injected intraperitoneally. Experiments were terminated after the last tumour-bearing mouse died and only mice free of detectable tumour remained at 60-80 days after tumour implantation. As controls, 1,079 tumour-implanted mice received no treatment. 10% of the controls spontaneously rejected the implanted tumour. The results are shown in Table 1.

TABLE 1
Havas's results sorted by mortality and regression.

| | | Number of | | |
|---|---|---|---|---|
| Formulation | Dosage mL | mice | % Mortality | % Regression |
| D14S7s | 0.01 | 48 | 0 | 62 |
| D4S2t | 0.01 | 16 | 0 | 44 |
| B4S2t | 0.05 | 86 | 2 | 41 |
| D4S2t | 0.05 | 56 | 3 | 54 |
| E4S2t | 0.05 | 105 | 3 | 41 |
| B14S7s | 0.05 | 80 | 6 | 30 |
| N4S2t | 0.05 | 31 | 10 | 52 |
| D14S7s | 0.05 | 56 | 11 | 39 |
| D4S2t | 0.10 | 24 | 12 | 29 |
| D14S7s | 0.10 | 28 | 14 | 50 |
| E14S7s | 0.05 | 408 | 14 | 43 |
| N14S7s | 0.05 | 32 | 22 | 50 |
| N28S7s | 0.05 | 32 | 31 | 38 |
| B14S7t | 0.05 | 34 | 38 | 44 |
| N28S7t | 0.05 | 32 | 44 | 37 |
| N14S7t | 0.05 | 32 | 66 | 31 |

% mortality = the percentage of mice that died within 72 hours of treatment;
% regression = the percentage of mice alive and free of tumour when the last tumour-bearing mouse died

The Havas experiments in mice confirm the clinical observations in humans that Multi Bacterial Vaccines can display wide variations in potency and therapeutic efficacy, depending upon formulation.

The immune stimulatory constituents of bacteria include DNA, lipopolysaccharide, peptidoglycan, lipoteichoic acid, streptolysin O, cytoplasmic membrane-associated protein, histone-like protein A, and exotoxins. Current immunological theory teaches that a therapeutic immune response can be initiated through stimulation of the immune system by the bacterial substances contained in a Multi Bacterial Vaccine (Hoption Cann, 2003; Matzinger, 1998). The mechanisms by which bacterial substances induce an immune response are also known. These mechanisms include binding cell surface receptors and thereby triggering the production of cytokines and chemokines, and stimulating the proliferation of immune system cells. However, prior to the present invention, there has not been a consistent method for standardizing, reproducing, and improving the efficacy of multibacterial vaccines.

### SUMMARY OF THE INVENTION

As described herein, the inventor has developed methodologies for characterizing and establishing standards for bacterial cultures, by determining the relative concentrations of immunostimulatory bacterial substances in the bacterial cultures. The inventor has further developed methods for reproducing previously-characterized bacterial cultures, for normalizing characterized bacterial cultures, for formulating characterized multibacterial vaccines composed of whole-cell lysates of characterized bacterial cultures, and for inhibiting and/or preventing disease by administering characterized multibacterial vaccine.

In addition, the inventor has developed multibacterial vaccines, composed of whole-cell lysates of Gram-negative and Gram-positive bacteria, in which the relative concentrations of at least four immunostimulatory bacterial substances are known. For example, the inventor has developed and reproduced Coley vaccines, composed of Gram-negative Serratia marcescens, in which the relative concentrations of Gram-negative DNA, lipopolysaccahride, and peptidoglycan are defined, and Gram-positive Streptococcus pyogenes, in which the relative concentrations of Gram-positive DNA, lipoteichoic acid and peptidoglycan are defined.

Accordingly, in one aspect, the present invention provides a method for establishing a standard for a Gram-negative bacterial culture, by determining the relative concentrations of at least two immunostimulatory bacterial substances (e.g., bacterial DNA, peptidoglycan, lipopolysaccharide, etc.) in the culture. In one embodiment, the relative concentrations of bacterial DNA, peptidoglycan, and lipopolysaccharide in the culture are determined. In another embodiment, the Gram-negative bacterial culture includes Serratia marcescens.

In another aspect, the present invention provides a method for establishing a standard for a Gram-positive bacterial culture, by determining the relative concentrations of at least two immunostimulatory bacterial substances (e.g., bacterial DNA, peptidoglycan, lipoteichoic acid, etc.) in the culture. In one embodiment, the relative concentrations of bacterial DNA, peptidoglycan, and lipoteichoic acid in the culture are determined. In another embodiment, the Gram-positive bacterial culture includes Streptococcus pyogenes.

In a further aspect, the present invention provides a method for establishing a standard for a mixed bacterial culture (e.g., a bacterial culture that includes at least one Gram-negative bacterium and at least one Gram-positive bacterium) by determining the relative concentrations of at least two immunostimulatory bacterial substances (e.g., Gram-negative bacterial DNA, Gram-positive bacterial DNA, peptidoglycan, lipopolysaccharide, lipoteichoic acid, etc.) in the mixed bacterial culture. In one embodiment, the relative concentrations of Gram-negative bacterial DNA, Gram-positive bacterial DNA, peptidoglycan, lipopolysaccharide, and lipoteichoic acid in the mixed bacterial culture are determined. In another embodiment, the mixed bacterial culture comprises a Coley vaccine.

In yet another aspect, the present invention provides a method for reproducing a Gram-negative bacterial culture, by: (a) obtaining a first Gram-negative bacterial culture; (b) determining the relative concentrations of at least two immunostimulatory bacterial substances (e.g., bacterial DNA, peptidoglycan, lipopolysaccharide, etc.) in the first culture; (c) obtaining a second Gram-negative bacterial culture; (d) determining the relative concentrations of the at least two immunostimulatory bacterial substances in the second culture; and (e) normalizing the second Gram-negative bacterial culture. In one embodiment, the relative concentrations of bacterial DNA, peptidoglycan, and lipopolysaccharide in the first culture and in the second culture are determined. In another embodiment, the method includes the step of determining the degree of equivalence between the normalized second culture and the first culture.

In still another aspect, the present invention provides a method for reproducing a Gram-positive bacterial culture, by: (a) obtaining a first Gram-positive bacterial culture; (b) determining the relative concentrations of at least two immunostimulatory bacterial substances (e.g., bacterial DNA, peptidoglycan, lipoteichoic acid, etc.) in the first culture; (c) obtaining a second Gram-positive bacterial culture; (d) determining the relative concentrations of the at least two immunostimulatory bacterial substances in the second culture; and (e) normalizing the second Gram-positive bacterial culture. In one embodiment, the relative concentrations of bacterial DNA, peptidoglycan, and lipoteichoic acid in the first culture and in the second culture are determined. In another embodiment, the method includes the step of determining the degree of equivalence between the normalized second culture and the first culture.

In a further aspect, the present invention provides a method for reproducing a mixed bacterial culture that includes at least one Gram-negative bacterium and at least one Gram-positive bacterium, by: (a) obtaining a first mixed bacterial culture; (b) determining the relative concentrations of at least two immunostimulatory bacterial substances (e.g., Gram-negative bacterial DNA, Gram-positive bacterial DNA, peptidoglycan, lipopolysaccharide, lipoteichoic acid, etc.) in the first culture; (c) obtaining a second mixed bacterial culture; (d) determining the relative concentrations of the at least two immunostimulatory bacterial substances in the second culture; and (e) normalizing the second mixed bacterial culture. In one embodiment, the relative concentrations of Gram-negative bacterial DNA, Gram-positive bacterial DNA, peptidoglycan, lipopolysaccharide, and lipoteichoic acid in the first culture and in the second culture are determined. In another embodiment, the method includes the step of determining the degree of equivalence between the normalized second culture and the first culture.

In yet another aspect, the present invention provides a method for preparing a multibacterial vaccine, by: (a) obtaining a Gram-negative bacterial culture; (b) determining the relative concentrations of bacterial DNA, peptidoglycan, and lipopolysaccharide in the Gram-negative bacterial culture; (c) obtaining a Gram-positive bacterial culture; (d) determining the relative concentrations of bacterial DNA, peptidoglycan, and lipoteichoic acid in the Gram-positive bacterial culture; and (e) combining the Gram-negative bacterial culture and the Gram-positive bacterial culture. Also provided is a multibacterial vaccine prepared in accordance with this method. The present invention further provides a method for treating and/or preventing a disorder (e.g., a burn, an infection, neoplasia, or a radiation injury) in a subject, by administering to the subject an amount of the multibacterial vaccine effective to treat and/or prevent the disorder in the subject.

In still another aspect, the present invention provides a method for preparing a multibacterial vaccine, by: (a) obtaining a mixed bacterial culture that includes a Gram-negative bacterial culture and a Gram-positive bacterial culture; and (b) determining the relative concentrations of Gram-negative bacterial DNA, Gram-positive bacterial DNA, peptidoglycan, lipopolysaccharide, and lipoteichoic acid in the mixed bacterial culture. Also provided is a multibacterial vaccine prepared in accordance with this method. The present invention further provides a method for treating and/or preventing a disorder (e.g., a burn, an infection, neoplasia, or a radiation injury) in a subject, by administering to the subject an amount of the multibacterial vaccine effective to treat and/or prevent the disorder in the subject.

In a further aspect, the present invention provides a method for predicting the efficacy of a multibacterial vaccine, by: (a) obtaining a first multibacterial vaccine having efficacy in the treatment and/or prevention of at least one disorder; (b) determining the relative concentrations of at least two immunostimulatory bacterial substances (e.g., Gram-negative bacterial DNA, Gram-positive bacterial DNA, peptidoglycan, lipopolysaccharide, lipoteichoic acid, etc.) in the first multibacterial vaccine; (c) obtaining a second multibacterial vaccine; (d) determining the relative concentrations of the at least two immunostimulatory bacterial substances in the second multibacterial vaccine; and (e) comparing the relative concentrations in the second multibacterial vaccine with the relative concentrations in the first multibacterial vaccine, wherein the second multibacterial vaccine is more efficacious if the relative concentrations in the second multibacterial vaccine are more similar to the relative concentrations in the first multibacterial vaccine, and wherein the second multibacterial vaccine is less efficacious if the relative concentrations in the second multibacterial vaccine are less similar to the relative concentrations in the first multibacterial vaccine. In one embodiment, the relative concentrations of Gram-negative bacterial DNA, Gram-positive bacterial DNA, peptidoglycan, lipopolysaccharide, and lipoteichoic acid in the first vaccine and in the second vaccine are determined. In another embodiment, the first multibacterial vaccine is a Coley vaccine. In yet another embodiment, the disorder is a bum, an infection, neoplasia, or a radiation injury.

In still another aspect, the present invention provides a method for enhancing the efficacy of a multibacterial vaccine, by: (a) obtaining a first multibacterial vaccine having efficacy in the treatment and/or prevention of at least one disorder; (b) determining the relative concentrations of at least two immunostimulatory bacterial substances (e.g., Gram-negative bacterial DNA, Gram-positive bacterial DNA, peptidoglycan, lipopolysaccharide, lipoteichoic acid, etc.) in the first multibacterial vaccine; (c) obtaining a second multibacterial vaccine; (d) determining the relative concentrations of the at least two immunostimulatory bacterial substances in the second culture; and (e) normalizing the second multibacterial vaccine. In one embodiment, the relative concentrations of Gram-negative bacterial DNA, Gram-positive bacterial DNA, peptidoglycan, lipopolysaccharide, and lipoteichoic acid in the first vaccine and in the second vaccine are determined. In another embodiment, the first multibacterial vaccine is a Coley vaccine. In still another embodiment, the disorder is a bum, an infection, neoplasia, or a radiation injury.

Additional aspects of the present invention will be apparent in view of the description which follows.

### DETAILED DESCRIPTION OF THE INVENTION

The present invention provides methodologies for characterizing bacterial cultures by determining the relative concentrations of immunostimulatory bacterial substances, for reproducing previously-characterized bacterial cultures, for normalizing characterized bacterial cultures, for formulating characterized multibacterial vaccines composed of whole-cell lysates of characterized bacterial cultures, and for preventing or inhibiting disease by administration of a characterized multibacterial vaccine.

For example, the present invention provides a method for characterizing a Gram-negative bacterial culture by determining the concentrations of bacterial DNA, peptidoglycan, and lipopolysaccharide. The present invention further provides a method for characterizing a Gram-positive bacterial culture by determining the concentrations of bacterial DNA, peptidoglycan, and lipoteichoic acid. The present invention also provides a method for characterizing a mixed bacterial culture containing Gram-negative and Gram-positive bacteria, by determining the concentrations of Gram-negative bacterial DNA, Gram-positive bacterial DNA, peptidoglycan, lipopolysaccharide, and lipoteichoic acid.

In addition, the present invention provides a method for reproducing a previously-characterized Gram-negative or Gram-positive bacterial culture by obtaining a new bacterial culture, determining the composition of the new bacterial culture using one of the methods described herein, normalizing the new bacterial culture, and confirming that the normalized new bacterial culture is equivalent to the original bacterial culture. Also provided is a method for reproducing a previously-characterized mixed bacterial culture by obtaining a new mixed bacterial culture, determining the composition of the new mixed bacterial culture in accordance with methods described herein, normalizing the new mixed bacterial culture, and confirming that the normalized new mixed bacterial culture is equivalent to the original mixed bacterial culture.

The present further provides a method for formulating a characterized multibacterial vaccine by combining a characterized Gram-negative bacterial culture with a characterized Gram-positive bacterial culture. In one embodiment, the method includes at least one of the following additional steps: lysing, lyophilizing, and reconstituting with a pharmaceutically-acceptable carrier, excipient, or diluent. The present invention also provides a method for preventing and/or inhibiting a disease state in a warm-blooded animal by administering a therapeutically-effective amount of a characterized multibacterial vaccine.

In preferred embodiments of the present invention, bacterial cultures are characterized by determining the concentrations of bacterial DNA, lipopolysaccharide, lipoteichoic acid, and peptidoglycan; previously-characterized bacterial cultures are reproduced and validated; multibacterial vaccines are formulated from characterized bacterial cultures; and disease states are inhibited by administration of a characterized multibacterial vaccine.

Previously-characterized bacterial cultures may be reproduced by growing new cultures in a standardized medium, from standardized bacterial seed stocks, under defined growth conditions including time, temperature, and exposure to light. However, because small changes in growth conditions can significantly impact concentrations of bacterial constituents, each culture batch should be validated by determining that the relative concentrations of immunostimulatory substances are within tolerance.

A characterized multibacterial vaccine can be formulated by combining characterized Gram-negative and Gram-positive bacterial cultures, and then lysing by heat sterilization, ultrasonication, mechanical agitation, or other procedures known to those skilled in the art. A characterized multibacterial vaccine may also be formulated by lysing a characterized mixed bacterial culture. The present invention further provides a method for treating or preventing a disease in a subject by administering to the subject the characterized multibacterial vaccine of the invention. For example, disease states in warm-blooded animals are prevented or inhibited by administering a therapeutically-effective amount of the characterized multibacterial vaccine.

More particularly, the present invention provides a method for establishing a standard for a Gram-negative bacterial culture. As used herein, the phrase “establishing a standard” includes setting a basis for comparison or a reference point against which other bacterial cultures may be compared. The method includes the step of determining the relative concentrations of at least two (e.g., 2, 3, etc.) immunostimulatory bacterial substances in the culture. As further used herein, the “relative concentration” of a substance is a reproducible determination that is proportional to the absolute concentration. In a Gram-negative bacterial culture, exemplary immunostimulatory bacterial substances include, without limitation, bacterial DNA, peptidoglycan, and lipopolysaccharide.

Bacterial DNA contains unmethylated CpG sequences that bind to the human Toll-like receptor, TLR9, and trigger an innate immune response that leads to the secretion of IL-6, IL-10, IL-12, IP-10, TNF-alpha, IFN-alpha, IFN-beta, and IFN-gamma (Coley Pharmaceuticals, 2003). In a bacterial culture, the concentration of each species of bacterial DNA may be determined by multiplying the number of bacteria of each species per unit volume times the genome size of each bacterial species. Procedures to determine the number of bacteria of each species per unit volume (e.g., use of a 1000× oil immersion microscope to directly count the number of bacteria in a counting chamber of known volume) are well known to those skilled in the art and described herein. The concentration of bacterial DNA may also be determined through comparative spectrographic measurements of the absorption of light of a suitable wavelength (e.g., 600 nm), by determination of the number of viable bacteria per unit volume (e.g., using a spiral plater), or by other methods known to those skilled in the art.

Peptidoglycan is a major component of the cell walls of Gram-positive bacteria, and a lesser component of gram-negative bacteria. Peptidoglycan induces cells to secrete TNF-alpha, IL-8, IL-1, and IL-6 (Dziarski, 1998; Wang, 2001; Schwandner, 1999). Peptidoglycan is a B-cell mitogen and a polyclonal activator in mice (Dziarski, 1982). The concentration of peptidoglycan in a bacterial culture can be determined by measuring the amount of the peptidoglycan-rich extract prepared by the Boiling Sodium Dodecyl Sulfate Procedure (de Jonge, 1992), or by other preparatory procedures and analytical techniques known to those skilled in the art.

Lipopolysaccharide (LPS) activates cells through the pattern-recognition receptors, CD14 and Toll-like receptor 2 (TLR2), on monocytes, macrophages, endothelium, and polymorphonuclear neutrophils, thereby inducing the release of TNF-alpha, IL-6, and nitric oxide (Dziarski, 1998; Matsuura, 1999). Nitric oxide is cytostatic and/or cytolytic for tumour cells (Farias-Eisner, 1994). Lipopolysaccharide also induces the production of IL-1-alpha, IL-8, IL-10, and small quantities of TNF-beta, and activates the complement pathway (Bjork, 1992; Hackett, 1993; Loos, 1986; Luster, 1996). Lipopolysaccharide is a B-cell mitogen and a polyclonal activator in mice (Dziarski, 1982). The concentration of lipopolysaccharide in a bacterial culture can be determined by measuring the amount of the lipopolysaccharide-rich extract prepared by the Phenol/Water Procedure (Galanos, 1979; Luchi, 2000), or by other preparatory procedures and analytical techniques known to those skilled in the art.

In one embodiment of the present invention, the relative concentrations of bacterial DNA, peptidoglycan, and lipopolysaccharide in the Gram-negative bacterial culture are determined. Exemplary Gram-negative bacteria for use in the present invention include, without limitation, Serratia marcescens.

The present invention also provides a method for establishing a standard for a Gram-positive bacterial culture. The method includes the step of determining the relative concentrations of at least two (e.g., 2, 3, etc.) immunostimulatory bacterial substances in the culture. In a Gram-positive bacterial culture, exemplary immunostimulatory bacterial substances include, without limitation, bacterial DNA, peptidoglycan, and lipoteichoic acid. In one embodiment of the present invention, the relative concentrations of bacterial DNA, peptidoglycan, and lipoteichoic acid in the culture are determined. Exemplary Gram-positive bacteria for use in the present invention include, without limitation, Streptococcus pyogenes.

Lipoteichoic acid binds to CD14 (Dziarski, 1998), inducing release of TNF. Lipoteichoic acid induces TNF-alpha, IFN-alpha, IFN-beta, and IFN-gamma in primed mice (Tsutsui, 1991); IL-1-beta, IL-6, and TNF in human monocyte cultures (Bhakdi, 1991; Keller, 1992; Yamamoto, 1985); IL-8 and MIP-1-alpha (Gao, 2001); and IL-12 (Cleveland, 1996). Lipoteichoic acid stimulates mitogenesis of T, but not B, lymphocytes (Beachey, 1979), and activates the complement pathway (Loos, 1986). The concentration of lipoteichoic acid in a bacterial culture can be determined by measuring the amount of the lipoteichoic-acid-rich extract prepared by the Aqueous Phenol Procedure (Fischer, 1983), or by other preparatory procedures and analytical techniques known to those skilled in the art.

The present invention further provides a method for establishing a standard for a mixed bacterial culture (e.g., a bacterial culture that includes at least one Gram-negative bacterium and at least one Gram-positive bacterium). The method of the invention includes the step of determining the relative concentrations of at least two immunostimulatory bacterial substances in the mixed bacterial culture. In a mixed bacterial culture, exemplary immunostimulatory bacterial substances include, without limitation, Gram-negative bacterial DNA, Gram-positive bacterial DNA, peptidoglycan, lipopolysaccharide, and lipoteichoic acid. In one embodiment, the relative concentrations of Gram-negative bacterial DNA, Gram-positive bacterial DNA, peptidoglycan, lipopolysaccharide, and lipoteichoic acid in the mixed bacterial culture are determined. Exemplary mixed bacterial cultures include, without limitation, Coley vaccines and other multibacterial vaccines.

In addition to bacterial DNA, peptidoglycan, lipopolysaccharide, and lipoteichoic acid, bacterial cultures may also contain a number of additional immunostimulatory bacterial substances. For example, bacterial cultures may also contain streptolysin O, cytoplasmic membrane-associated protein, histone-like protein A, and exotoxins.

Streptolysin O stimulates monocytes to produce IL-1-beta and TNF-alpha (Hackett, 1992), and stimulates bone-marrow-derived mast cells to produce IL-4, IL-6, IL-13, GM-CSF, TNF-alpha, and MCP-1 (Stassen, 2003). It also binds IgG antibodies to form immune complexes with potent complement-activating capacity (Bhakdi, 1985).

Cytoplasmic membrane-associated protein stimulates polyclonal activation of many classes of T lymphocytes (Itoh, 1992).

Histone-like protein A stimulates macrophages to produce TNF-alpha and IL-1 (Zhang, 1999).

Exotoxins are extracellular toxins secreted into their environment by Gram-positive bacteria. Exotoxins are both pyrogenic (induce fever) and mitogenic (induce cellular proliferation). They are pyrogenic because they stimulate the production of cytokines and chemokines; they are mitogenic because they function as “superantigens” which can give rise to polyclonal activation (Marrack, 1990; Leonard, 1991). Superantigens have the ability to bind major histocompatibility complex molecules on antigen-presenting cells and, simultaneously, T cell receptors, thereby triggering a polyclonal expansion of T lymphocytes.

The best-known exotoxins are the streptococcal pyrogenic exotoxins (Spe), which are produced in the cell walls of group A streptococci and secreted into the extracellular environment. These exotoxins include SpeA, SpeB, SpeC, and a number of other exotoxins, including SpeF, SpeG, Spel, SpeJ, SpeZ, SSA, SMEZ, and SMEZ-2. The best-characterized streptococcal pyrogenic exotoxin is SpeA. SpeA stimulates the production of cytokines IL-1-alpha, IL-6, TNF-alpha, IL-12, IL-10, and IP-10; Thl-derived cytokines TNF-beta, IFN-gamma, and IL-2; Th2-derived cytokine IL-5; IL-3 and GM-CSF; and chemokines IL-8, RANTES, and MIP-1-alpha (Muller-Alouf, 2001).

The present invention further provides a method for reproducing a Gram-negative bacterial culture. As used herein, the term “reproducing” includes duplicating, making a copy of, or making an equivalent of a bacterial culture. The method of the invention includes the steps of: (a) obtaining a first Gram-negative bacterial culture; (b) determining the relative concentrations of at least two immunostimulatory bacterial substances in the first culture; (c) obtaining a second Gram-negative bacterial culture; (d) determining the relative concentrations of the same immunostimulatory bacterial substances in the second culture; and (e) normalizing the second Gram-negative bacterial culture. As used herein, the term “normalizing” means bringing a second bacterial culture into conformity with a first (or standard) bacterial culture, by adjusting the relative concentrations of bacterial substances in the second bacterial culture to conform with the relative concentrations of those same bacterial substances in the first (or standard) bacterial culture.

In one embodiment of the present invention, the relative concentrations of bacterial DNA, peptidoglycan, and lipopolysaccharide in the first culture and in the second culture are determined. In another embodiment, the method further includes the step of determining the degree of equivalence between the normalized second culture and the first culture (e.g., by determining the accuracy with which the relative concentrations of the second culture reproduce the relative concentrations of the first culture). By way of example, the second culture may be normalized, relative to the first culture, through dilution or evaporation. The relative concentrations of immunostimulatory bacterial substances in the second culture may then be assessed to confirm that they are within tolerance of the relative concentrations of the same immunostimulatory bacterial substances in the first culture. In one embodiment, the relative concentrations of the second culture are defined to an accuracy of at least 10%, as compared with the relative concentrations of the first culture.

The present invention also provides a method for reproducing a Gram-positive bacterial culture. The method includes the steps of: (a) obtaining a first Gram-positive bacterial culture; (b) determining the relative concentrations of at least two immunostimulatory bacterial substances in the first culture; (c) obtaining a second Gram-positive bacterial culture; (d) determining the relative concentrations of the same immunostimulatory bacterial substances in the second culture; and (e) normalizing the second Gram-positive bacterial culture. In one embodiment of the present invention, the relative concentrations of bacterial DNA, peptidoglycan, and lipoteichoic acid in the first culture and in the second culture are determined. In another embodiment, the method further includes the step of determining the degree of equivalence between the normalized second culture and the first culture.

Additionally, the present invention provides a method for reproducing a mixed bacterial culture (e.g., a bacterial culture that includes at least one Gram-negative bacterium and at least one Gram-positive bacterium). The method includes the steps of: (a) obtaining a first mixed bacterial culture; (b) determining the relative concentrations of at least two immunostimulatory bacterial substances in the first culture; (c) obtaining a second mixed bacterial culture; (d) determining the relative concentrations of the same immunostimulatory bacterial substances in the second culture; and (e) normalizing the second mixed bacterial culture. In one embodiment of the present invention, the relative concentrations of Gram-negative bacterial DNA, Gram-positive bacterial DNA, peptidoglycan, lipopolysaccharide, and lipoteichoic acid in the first culture and in the second culture are determined. In another embodiment, the method further includes the step of determining the degree of equivalence between the normalized second culture and the first culture.

The present invention further provides a method for preparing a multibacterial vaccine. As used herein, a “multibacterial vaccine” is a vaccine that includes at least one Gram-negative bacterium and at least one Gram-positive bacterium. Exemplary multibacterial vaccines include, without limitation, Coley vaccines. As further used herein, a “vaccine” is a preparation that includes an antigen (e.g., any molecule against which a host is capable of mounting an immune response, including a molecule that confers immunity against a disorder). The antigen may include whole disease-causing organisms (killed or weakened) or parts thereof.

In accordance with the method of the present invention, a multibacterial vaccine may be prepared by: (a) obtaining a Gram-negative bacterial culture; (b) determining the relative concentrations of bacterial DNA, peptidoglycan, and lipopolysaccharide in the Gram-negative bacterial culture; (c) obtaining a Gram-positive bacterial culture; (d) determining the relative concentrations of bacterial DNA, peptidoglycan, and lipoteichoic acid in the Gram-positive bacterial culture; and (e) combining the Gram-negative bacterial culture with the Gram-positive bacterial culture. Also provided is a multibacterial vaccine prepared in accordance with this method. In one embodiment, the method optionally includes at least one of the following additional steps: (f) lysing the combined bacterial cultures; (g) lyophilizing the lysed bacterial cultures; and (h) reconstituting the lyophilized bacterial cultures with a pharmaceutically-acceptable carrier, diluent, or excipient.

A vaccine of the present invention may be prepared in accordance with methods well-known in the pharmaceutical arts. For example, the vaccine may be brought into association with a pharmaceutically-acceptable carrier, excipient, or diluent, such as a suspension or solution. The carrier, excipient, or diluent must be “acceptable” in the sense of being compatible with the other ingredients of the composition, and not deleterious to the recipient thereof. The pharmaceutically-acceptable carrier, excipient, or diluent employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavour-improving agents, preservatives, and sweeteners, may also be added. Examples of acceptable pharmaceutical carriers, excipients, or diluents include carboxymethyl cellulose, crystalline cellulose, glycerin, gum arabic, lactose, magnesium stearate, methyl cellulose, powders, saline, sodium alginate, sucrose, starch, talc, and water, among others. Optionally, one or more accessory ingredients (e.g., buffers, flavouring agents, surface active agents, and the like) also may be added to the multibacterial vaccine preparation of the invention.

The choice of carrier, excipient, or diluent will also depend upon the route of administration of the vaccine. Formulations of the vaccine may be conveniently presented in unit dosage, or in such dosage forms as aerosols, capsules, elixirs, emulsions, eye drops, injections, liquid drugs, pills, powders, granules, suppositories, suspensions, syrup, tablets, or troches, which can be administered orally, topically, or by injection, including, but not limited to, intravenous, intraperitoneal, subcutaneous, intramuscular, and intratumoural (i.e., direct injection into a tumour) injection.

The multibacterial vaccine of the present invention may be used to trigger an immune response in a subject. The nature of the immune response will vary, depending upon the particular immunostimulatory bacterial substances included in the vaccine.

For example, the nature of the immune response triggered by a bacterial CpG DNA sequence depends on the level of homology to the optimal human TLR9-CpG motif of GTCGTT (Bauer, 2001). Since each bacterial species has a uniquely-sized genome incorporating different numbers and varieties of CpG DNA sequences, the immunostimulatory properties of Gram-negative and Gram-positive bacteria are qualitatively different.

Induction of the maturation of the largest population of dendritic cells requires a combination of bacterial substances. Both CD4-positive and CD4-negative peripheral blood dendritic precursor cells respond to CpG DNA, but these dendritic cells show little response to lipopolysaccharide. In contrast, monocyte-derived dendritic cells do not respond to CpG DNA, but are highly sensitive to lipopolysaccharide (Hartmann, 1999).

The relative concentrations of lipopolysaccharide and peptidoglycan also influence the complexity of the immune response. Lipopolysaccharide binds to the receptor CD14, and induces the release of TNF (Dziarski, 1998); however, peptidoglycan (which also induces TNF) interacts via a different receptor, because blockage of CD14 has no influence on peptidoglycan-induced TNF (Wang, 2000). Even so, lipopolysaccharide can partially block the induction of other monokines by peptidoglycan (Weidemann, 1994).

Peptidoglycan from Gram-negative bacteria is different from peptidoglycan from Gram-positive bacteria, because the two types of peptidoglycan can stimulate the immune system via different pathways. In the fruit fly, for example, peptidoglycan from Gram-negative bacteria induces an immune response primarily through the lmd pathway, and peptidoglycan from Gram-positive bacteria induces an immune response primarily through the Toll pathway (Leulier, 2003).

The immune responses triggered by lipoteichoic acid and peptidoglycan can be profoundly different. In mice, lipoteichoic acid suppresses Meth A fibrosarcoma tumour growth, but peptidoglycan does not. Moreover, in mice primed with Propionibacterium acnes, lipoteichoic acid induces TNF, but peptidoglycan does not (Usami, 1988).

The multibacterial vaccine of the present invention may also be useful for treating and/or preventing a disorder in a subject. Accordingly, the present invention further provides a method for treating and/or preventing a disorder in a subject, by administering to the subject a multibacterial vaccine of the invention. As used herein, the “subject” is a bird (e.g., a chicken, turkey, etc.) or a mammal (e.g., a cow, dog, human, monkey, mouse, pig, rat, etc.). In one embodiment, the subject is a human. The multibacterial vaccine is administered to a subject in an amount effective to treat and/or prevent the disorder in the subject. This amount may be readily determined by the skilled artisan.

Exemplary disorders which may be treated and/or prevented by the multibacterial vaccine of the present invention include, without limitation, burns, infections, neoplasia, and radiation injuries. In one embodiment, the disorder is neoplasia. As used herein, the term “neoplasia” refers to the uncontrolled and progressive multiplication of tumour cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumour cells having the unique trait of loss of normal controls, resulting in unregulated growth, lack of differentiation, local tissue invasion, and/or metastasis.

Exemplary neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumours and malignant tumours (e.g., colon tumours) that are either invasive or non-invasive. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumours of the central nervous system (e.g., gliomas, astrocytomas, oligodendrogliomas, ependymomas, glioblastomas, neuroblastomas, ganglioneuromas, gangliogliomas, medulloblastomas, pineal cell tumours, meningiomas, meningeal sarcomas, neurofibromas, and Schwannomas); germ-line tumours (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumours of mixed origin, such as Wilms' tumour and teratocarcinomas (Beers and Berkow (eds.), 1999).

In accordance with the method of the present invention, a multibacterial vaccine as described herein may be administered to a subject who has a disorder, in an amount effective to treat the disorder in the subject. As used herein, the phrase “effective to treat the disorder” means effective to ameliorate or minimize the clinical impairment or symptoms resulting from the disorder. For example, where the subject has neoplasia, the clinical impairment or symptoms of the neoplasia may be ameliorated or minimized by diminishing any pain or discomfort suffered by the subject; by extending the survival of the subject beyond that which would otherwise be expected in the absence of such treatment; by inhibiting or preventing the development or spread of the neoplasia; and/or by limiting, suspending, terminating, or otherwise controlling the proliferation of cells in the neoplasm.

The amount of multibacterial vaccine effective to treat a disorder in a subject will vary depending on the particular factors of each case, including the subject's weight and the severity of the subject's condition. For example, the amount of multibacterial vaccine that is effective to treat neoplasia in a subject will vary depending on the particular factors of each case, including the type of neoplasia, the stage of neoplasia, the subject's weight, the severity of the subject's condition, and the method of administration. The appropriate effective amount of multibacterial vaccine can be readily determined by the skilled artisan.

In the method of the present invention, a multibacterial vaccine of the invention may also be administered to a subject at risk of developing a disorder, in an amount effective to prevent the disorder in the subject. As used herein, the phrase “effective to prevent the disorder” includes effective to hinder or prevent the development or manifestation of clinical impairment or symptoms resulting from the disorder. The amount of multibacterial vaccine effective to prevent a disorder in a subject will vary depending on the particular factors of each case, including the subject's weight and the severity of the subject's condition. The appropriate amount of multibacterial vaccine can be readily determined by the skilled artisan.

The multibacterial vaccine of the invention may be administered to a human or animal subject by known procedures, including, without limitation, oral administration, parenteral administration (e.g., epifascial, intracapsular, intracutaneous, intradermal, intramuscular, intraorbital, intraperitoneal, intraspinal, intrasternal, intravascular, intravenous, parenchymatous, or subcutaneous administration), transdermal administration, intranasal administration, pulmonary administration (e.g., intratracheal administration), and administration by osmotic pump. In one embodiment, the method of administration is parenteral administration, by intravenous or subcutaneous injection.

For oral administration, the formulation of the multibacterial vaccine may be presented as capsules, tablets, powders, granules, or as a suspension. The formulation may have conventional additives, such as lactose, mannitol, corn starch, or potato starch. The formulation also may be presented with binders, such as crystalline cellulose, cellulose derivatives, acacia, corn starch, or gelatins. Additionally, the formulation may be presented with disintegrators, such as corn starch, potato starch, or sodium carboxymethylcellulose. The formulation may be further presented with dibasic calcium phosphate anhydrous or sodium starch glycolate. Finally, the formulation may be presented with lubricants, such as talc or magnesium stearate.

For parenteral administration, the multibacterial vaccine may be combined with a sterile aqueous solution, which is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampoules or vials. The formulation also may be delivered by any mode of injection, including any of those described herein.

For transdermal administration, the multibacterial vaccine may be combined with skin penetration enhancers, such as propylene glycol, polyethylene glycol, isopropanol, ethanol, oleic acid, N-methylpyrrolidone, and the like, which increase the permeability of the skin to the multibacterial vaccine, and permit the multibacterial vaccine to penetrate through the skin and into the bloodstream. The composition of enhancer and multibacterial vaccine also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The multibacterial vaccine may be administered transdermally, at or near the site on the subject where the bum, infection, neoplasm, or other disorder may be localized. Alternatively, the multibacterial vaccine may be administered transdermally at a site other than the affected area, in order to achieve systemic administration.

For intranasal administration (e.g., nasal sprays) and/or pulmonary administration (administration by inhalation), formulations of the multibacterial vaccine, including aerosol formulations, may be prepared in accordance with procedures well known to persons of skill in the art. Aerosol formulations may comprise either solid particles or solutions (aqueous or non-aqueous). Nebulizers (e.g., jet nebulizers, ultrasonic nebulizers, etc.) and atomizers may be used to produce aerosols from solutions (e.g., using a solvent such as ethanol); metered-dose inhalers and dry-powder inhalers may be used to generate small-particle aerosols. The desired aerosol particle size can be obtained by employing any one of a number of methods known in the art, including, without limitation, jet-milling, spray drying, and critical-point condensation.

Pharmaceutical compositions for intranasal administration may be solid formulations (e.g., a coarse powder) and may contain excipients (e.g., lactose). Solid formulations may be administered from a container of powder held up to the nose, using rapid inhalation through the nasal passages. Compositions for intranasal administration may also comprise aqueous or oily solutions of nasal spray or nasal drops. For use with a sprayer, the formulation of multibacterial vaccine may comprise an aqueous solution and additional agents, including, for example, an excipient, a buffer, an isotonicity agent, a preservative, or a surfactant. A nasal spray may be produced, for example, by forcing a suspension or solution of the multibacterial vaccine through a nozzle under pressure.

Formulations of the multibacterial vaccine for pulmonary administration may be presented in a form suitable for delivery by an inhalation device, and may have a particle size effective for reaching the lower airways of the lungs or sinuses. For absorption through mucosal surfaces, including the pulmonary mucosa, the formulation of the present invention may comprise an emulsion that includes, for example, a bioactive peptide, a plurality of submicron particles, a mucoadhesive macromolecule, and/or an aqueous continuous phase. Absorption through mucosal surfaces may be achieved through mucoadhesion of the emulsion particles.

Pharmaceutical compositions for use with a metered-dose inhaler device may include a finely-divided powder containing the multibacterial vaccine as a suspension in a non-aqueous medium. For example, the multibacterial vaccine may be suspended in a propellant with the aid of a surfactant (e.g., sorbitan trioleate, soya lecithin, or oleic acid). Metered-dose inhalers typically use a propellant gas (e.g., a chlorofluorocarbon, a hydrochlorofluorocarbon, a hydrofluorocarbon, or a hydrocarbon) stored in a container (e.g., a canister) as a mixture (e.g., as a liquefied, compressed gas). Inhalers require actuation during inspiration. For example, actuation of a metering valve may release the mixture as an aerosol. Dry-powder inhalers use breath-actuation of a mixed powder.

The multibacterial vaccine of the present invention also may be released or delivered from an osmotic mini-pump or other timed-release device. The release rate from an elementary osmotic mini-pump may be modulated with a microporous, fast-response gel disposed in the release orifice. An osmotic mini-pump would be useful for controlling release, or targeting delivery, of the multibacterial vaccine.

The multibacterial vaccine of the present invention may be administered or introduced to a subject by known techniques used for the introduction of drugs, including, for example, injection and transfusion. Where a disorder is localized to a particular portion of the body of the subject, it may be desirable to introduce the multibacterial vaccine directly to that area by injection or by some other means (e.g., by introducing the multibacterial vaccine into the blood or another body fluid).

In accordance with the method of the present invention, the multibacterial vaccine may be administered to a subject who has a disorder, either alone or in combination with one or more drugs used to treat that disorder. For example, where the subject has neoplasia, the multibacterial vaccine of the invention may be administered to a subject in combination with at least one antineoplastic drug. Examples of antineoplastic drugs with which the multibacterial vaccine may be combined include, without limitation, carboplatin, cyclophosphamide, doxorubicin, etoposide, and vincristine. Additionally, when administered to a subject who suffers from neoplasia, the multibacterial vaccine may be combined with other neoplastic therapies, including, without limitation, surgical therapies, radiotherapies, gene therapies, and immunotherapies.

The present invention also provides a method for preparing a multibacterial vaccine, by: (a) obtaining a mixed bacterial culture comprising a Gram-negative bacterial culture and a Gram-positive bacterial culture; and (b) determining the relative concentrations of Gram-negative bacterial DNA, Gram-positive bacterial DNA, peptidoglycan, lipopolysaccharide, and lipoteichoic acid in the mixed bacterial culture. Also provided is a multibacterial vaccine prepared in accordance with this method. In one embodiment of the invention, the method further includes at least one of the following additional steps: (c) lysing the mixed bacterial culture; (d) lyophilizing the lysed bacterial culture; and (e) reconstituting the lyophilized bacterial culture with a pharmaceutically-acceptable carrier, diluent, or excipient.

The present invention further provides a method for treating and/or preventing a disorder in a subject, by administering to the subject a multibacterial vaccine prepared in accordance with the above-described method. The multibacterial vaccine may be administered in an amount effective to treat and/or prevent the disorder in the subject. Exemplary disorders which may be treated and/or prevented by the multibacterial vaccine of the present invention include, without limitation, a bum, an infection, neoplasia, and a radiation injury.

In addition, the present invention provides a method for predicting the efficacy of a multibacterial vaccine, by: (a) obtaining a first multibacterial vaccine having efficacy in the treatment and/or prevention of at least one disorder; (b) determining the relative concentrations of at least two (e.g., 2, 3, etc.) immunostimulatory bacterial substances in the first multibacterial vaccine; (c) obtaining a second multibacterial vaccine; (d) determining the relative concentrations of the same immunostimulatory bacterial substances in the second multibacterial vaccine; and (e) comparing the relative concentrations in the second multibacterial vaccine with the relative concentrations in the first multibacterial vaccine. The second multibacterial vaccine is more efficacious if the relative concentrations in the second multibacterial vaccine are more similar to the relative concentrations in the first multibacterial vaccine; the second multibacterial vaccine is less efficacious if the relative concentrations in the second multibacterial vaccine are less similar to the relative concentrations in the first multibacterial vaccine. In one embodiment, the relative concentrations of Gram-negative bacterial DNA, Gram-positive bacterial DNA, peptidoglycan, lipopolysaccharide, and lipoteichoic acid in the first vaccine and in the second vaccine are determined. In another embodiment, the first multibacterial vaccine is a Coley vaccine. In still another embodiment, the first multibacterial vaccine has efficacy in the treatment and/or prevention of a burn, an infection, neoplasia, and a radiation injury.

The present invention also provides a method for enhancing the efficacy of a multibacterial vaccine, by: (a) obtaining a first multibacterial vaccine having efficacy in the treatment and/or prevention of at least one disorder; (b) determining the relative concentrations of at least two (e.g., 2, 3, etc.) immunostimulatory bacterial substances in the first multibacterial vaccine; (c) obtaining a second multibacterial vaccine; (d) determining the relative concentrations of the same immunostimulatory bacterial substances in the second culture; and (e) normalizing the second multibacterial vaccine. In one embodiment, the relative concentrations of Gram-negative bacterial DNA, Gram-positive bacterial DNA, peptidoglycan, lipopolysaccharide, and lipoteichoic acid are determined. In another embodiment, the first multibacterial vaccine is a Coley vaccine. In yet another embodiment, the first multibacterial vaccine has efficacy in the treatment and/or prevention of a bum, an infection, neoplasia, and a radiation injury.

The present invention is described in the following Examples, which are set forth to aid in the understanding of the invention, and should not be construed to limit in any way the scope of the invention as defined in the claims which follow thereafter.

### EXAMPLES

### Example 1

### Characterizing a Gram-negative Bacterial Culture

A Gram-negative bacterial culture is prepared in accordance with the following method. 50 mL of neopeptone broth (10 g/L neopeptone (DIFCO, 0119-17), 3 g/L beef extract (Sigma, B4888), 5 g/L NaCl) is seeded with Serratia marcescens (ATCC, 8195) and grown at 25° C. on an orbital shaker (50 rpm through a 19-mm orbit) for 24 hours. The bacterial concentration of the seed stock is determined by the direct counting method, using a Neubauer counting chamber (VWR, 15170-081) and a 1000× oil immersion microscope. 1.5 L of neopeptone broth is seeded with an aliquot containing 10⁵ Serratia marcescens, and grown at 25° C. on an orbital shaker for 144 hours. The resulting culture, designated SM144A, is quickly chilled in an ice/ethanol bath, until the temperature drops below 10° C., and stored at 4° C. Using the direct counting method, the whole-genome Serratia marcescens DNA concentration of SM144A is determined at 6×10⁸ genomes per mL.

The concentration of peptidoglycan is determined by a modified de Jonge technique (de Jonge, 1992). Bacteria are harvested from 500 mL of SM144A culture by centrifugation at 15,000×g (10 min, 4° C.), and transferred into 4% (final concentration) boiling sodium dodecyl sulfate (SDS). The cells are boiled for 30 min. The cell walls are concentrated by centrifugation for 10 min at 30,000×g, and washed three times with double-distilled water. Cell walls are broken with glass beads (0.2 mm) on a Vortex at 3000 rpm and 4° C. for 15 min. The suspension is centrifuged at 2,000×g for 10 min; after removal of the supernatant, the pellet is again treated with glass beads as described above. The collected broken walls are centrifuged at 40,000×g for 15 min, and the pellet is treated at 37° C. in 100 mM Tris-HCI (pH 7.5) with alpha-amylase (100 μg/mL; Sigma, 10080). After 2 hours, DNase (10 μg/mL; Sigma, D4513) and RNase (50 μg/mL; Sigma, R4875) are added with 20 mM (final concentration) MgSO₄, and the incubation is prolonged for another 2 hours. Finally, the suspension is treated with trypsin (100 μg/mL; Sigma, T6763) in the presence of 10 mM CaCl₂ (final concentration) for 16 hours.

The enzymes are inactivated by boiling for 15 min in 1% (final concentration) SDS. The cell wall extract is centrifuged at 40,000×g for 15 min, and washed two times with double-distilled water—once with 8 M LiCl, and once with 100 mM EDTA—and two times with double-distilled water, before being washed with acetone. The resulting extract is resuspended in double-distilled water, lyophilized, and weighed. The concentration of the peptidoglycan-rich extract in SM144A is determined to be 0.95 μg/mL.

The concentration of lipopolysaccharide is determined by a modified Luchi phenol-water technique (Luchi, 2000). Bacteria are harvested from 200 mL of SM144A culture by centrifugation at 15,000×g (10 min, 4° C.), suspended in 50 mL double-distilled water, and extracted with an equal volume of 90% aqueous phenol at 68° C. two times. The combined aqueous extracts are dialyzed against ten volumes of double-distilled water at 4° C., and lyophilized. Nucleic acids are removed by reconstitution of the lipopolysaccharide-enriched extract to 10 mg/mL in 0.1 M acetate buffer with 0.02% MgSO₄ and 0.4% chloroform, and digestion with RNase (0.4 mg/mL; Sigma, R4875) and DNase (20 μg/mL; Sigma, D4513) by incubation at 37° C. for 12 hours. Contaminating protein is then removed by the addition of proteinase K (20 μg/mL; Sigma, P2308) in 0.1 M Tris (pH 8.0), followed by heating at 60° C. for 1 hour and then incubation for 12 hours at 37° C. The extract is then dialyzed against 250 mL of double-distilled water six times, lyophilized, and weighed. The concentration of the lipopolysaccharide-rich extract in SM144A is determined to be 8.60 μg/mL. The characterized SM144A Gram-negative bacterial culture contains 6.6×10⁸ genomes of bacterial DNA per mL, 0.95 μg of peptidoglycan-rich extract per mL, and 8.60 μg of lipopolysaccharide-rich extract per mL.

### Example 2

### Characterizing a Gram-positive Bacterial Culture

A Gram-positive bacterial culture is prepared in accordance with the following method. 50 mL of neopeptone broth (10 g/L neopeptone (DIFCO, 0119-17), 3 g/L beef extract (Sigma, B4888), 5 g/L NaCl) is seeded with Streptococcus pyogenes (ATCC, 12351) and grown at 37° C. on an orbital shaker (50 rpm through a 19 mm orbit) for 24 hours. The bacterial concentration of the seed stock is determined by the direct counting method described in Example 1. 1.5 L of neopeptone broth is seeded with an aliquot containing 10⁵ Streptococcus pyogenes, and grown at 37° C. on an orbital shaker for 288 hours. The resulting culture, designated SP288A, is quickly chilled in an ice/ethanol bath, until the temperature drops below 10° C., and stored at 4° C.

Using the direct counting method, the whole-genome Streptococcus pyogenes DNA concentration of SP288A is determined at 2×10⁷ genomes per mL. Using the modified de Jonge technique of Example 1, the concentration of the peptidoglycan-rich extract in SP288A is determined to be 4.60 μg/mL.

The concentration of lipoteichoic acid is determined by a modified Fischer technique (Fischer, 1983). Bacteria are harvested from 200 mL of SP288A culture by centrifugation at 2,000×g (15 min, 4° C.), and are suspended in 50 mL of 0.1 M sodium citrate (pH 4.7). The cell walls are broken with glass beads (0.2 mm) on a Vortex at 2,500 rpm and 4° C. for 15 min. The suspension is centrifuged at 2,000×g (15 min, 4° C.); after removal of the supernatant, the pellet is again treated with glass beads as described above. The suspension of broken cells is decanted, and the glass beads are washed with 10 mL of 0.1 M sodium citrate (pH 4.7). An equal volume of 80% (w/v) aqueous phenol is added, and the mixture is stirred at 65° C. for 1 hour. After cooling, the emulsion is centrifuged (3000×g for 30 min), and the aqueous layer is collected. The phenol layer and the insoluble residue are stirred with an equal volume of 0.1 M sodium acetate (pH 4.7), and centrifuged as before. The combined aqueous layers are dialyzed for 24 hours against four 5-1 changes of 0.1 M sodium acetate (pH 5.0). The extract is then dialyzed against 250 mL of double-distilled water six times, and lyophilized. Nucleic acids are removed by reconstitution of the lipoteichoic-acid-enriched extract to 10 mg/mL in 0.1 M acetate buffer with 0.02% MgSO₄ and 0.4% chloroform, and digestion with RNase (0.4 mg/mL; Sigma, R4875) and DNase (20 μg/mL; Sigma, D4513) by incubation at 37° C. for 12 hours. Contaminating protein is then removed by the addition of proteinase K (20 μg/mL; Sigma, P2308) in 0.1 M Tris (pH 8.0), followed by heating at 60° C. for 1 hour and incubation for 12 hours at 37° C. The extract is then dialyzed against 250 mL of double-distilled water six times, lyophilized, and weighed. The concentration of the lipoteichoic-acid-rich extract in SP288A is determined to be 3.9 μg/mL. The characterized SP288A Gram-positive bacterial culture contains 2.1×10⁷ genomes of bacterial DNA per mL, 4.62 μg of peptidoglycan-rich extract per mL, and 3.93 μg of lipoteichoic-acid-rich extract per mL.

### Example 3

### Characterizing a Mixed Bacterial Culture

A mixed bacterial culture is prepared in accordance with the following method. 1.5 L of neopeptone broth is seeded with an aliquot containing 10⁵ Streptococcus pyogenes, prepared as described in Example 2, and grown at 37° C. on an orbital shaker (50 rpm through a 19-mm orbit). After 96 hours, the temperature is reduced to 25° C., and the culture is inoculated with an aliquot containing 10⁵ Serratia marcescens, prepared as described in Example 1, and grown on an orbital shaker for 96 hours. The resulting culture, designated SM4SP8A, is quickly chilled in an ice/ethanol bath, until the temperature drops below 10° C., and stored at 4° C.

Using the direct counting method described in Example 1, the whole-genome DNA concentration of the rod-shaped Gram-negative bacteria Serratia marcescens in SM4SP8A is determined to be 1.7×10⁸ genomes per mL, and the whole-genome DNA concentration of the coccoid-shaped Gram-positive bacteria Streptococcus pyogenes in SM4SP8A is determined to be 5.8×10⁶ genomes per mL.

Using the modified de Jonge technique described in Example 1, the concentration of the peptidoglycan-rich extract in SM4SP8A is determined to be 1.15 μg/mL. Using the modified Lucci phenol-water technique described in Example 1, the concentration of the lipopolysaccharide-rich extract in SM4SP8A is determined to be 2.08 μg/mL. Using the modified Fischer technique described in Example 2, the concentration of the lipoteichoic-acid-rich extract in SM4SP8A is determined to be 1.22 μg/mL.

### Example 4

### Reproducing a Previously-characterized Gram-negative or Gram-positive Bacterial Culture

In this example, a new bacterial culture SM144B is considered equivalent to the previously-characterized bacterial culture SM144A if the concentration of each of the three measured substances in SM144B is within 10% of the corresponding concentration in SM144A.

A Gram-negative bacterial culture SM144B is prepared and characterized as described in Example 1. The whole-genome Serratia marcescens DNA concentration of SM144B is determined to be 5.5×10⁸ genomes per mL, the concentration of the peptidoglycan-rich extract is determined to be 0.88 μg/mL, and the concentration of lipopolysaccharide is determined to be 7.90 μg/mL.

Before comparing the two cultures, the concentrations of DNA, peptidoglycan, and lipopolysaccharide in SM144B are normalized to agree most closely with the corresponding concentrations in SM144A from Example 1 (which are, respectively, 6.6×10⁸ genomes per mL, 0.95 μg/mL, and 8.6 μg/mL). The normalization factor is the amount by which each of the three concentrations in SM144B must be adjusted through dilution or evaporation to obtain the culture most similar to SM144A.

The normalization factor is a function of the concentrations of the two substances in SM144B that deviate from the concentrations in SM144A by the largest and smallest amounts. Since the concentrations in SM144B differ from the concentrations in SM144A by −16.67% for DNA, −7.37% for peptidoglycan, and −8.14% for lipopolysaccharide, the two substances used to calculate the normalization factor are DNA and peptidoglycan. In the normalized SM144B culture, as compared with SM144A, the percentage deviations of DNA and peptidoglycan are identical, but of different signs, and the percentage deviation of lipopolysaccharide lies somewhere in between.

The normalization factor is calculated by solving the equation:
{([DNA2]×NF)/[DNA1]}+{([PGN2]×NF)/[PGN1]}=2
wherein NF is the normalization factor; [DNA1] is the DNA concentration in SM144A; [DNA2] is the DNA concentration in SM144B; [PGN1] is the peptidoglycan concentration in SM144A; and [PGN2] is the peptidoglycan concentration in SM144B.

The equation yields a normalization factor of 1.1366, meaning that normalization is accomplished by evaporation. In order to increase the concentration of each measured substance by a factor of 1.1366, the volume of the normalized culture must be equal to the reciprocal percentage of 1.1366, or 87.98% of the original volume.

In the present example, normalization of SM144B by a factor of 1.1366 yields a normalized SM144B culture that, as compared with SM144A, contains 5.28% less DNA, 5.28% more peptidoglycan, and 4.41% more lipopolysaccharide. Therefore, the two cultures are equivalent because the variation in concentration of each of the three measured substances is less than 10%.

### Example 5

### Reproducing a Previously-characterized Mixed Bacterial Culture

In this example, the new mixed bacterial culture SM4SP8B is considered equivalent to the previously-characterized mixed bacterial culture SM4SP8A if the concentration of each of the five measured substances in SM4SP8B is within 10% of the concentration of the corresponding substance in SM4SP8A.

The mixed bacterial culture SM4SP8B is prepared and characterized as described in Example 3. The whole-genome DNA concentration of the rod-shaped Gram-negative bacteria Serratia marcescens in SM4SP8B is determined to be 2.8×10⁸ genomes per mL, and the whole-genome DNA concentration of the coccoid-shaped Gram-positive bacteria Streptococcus pyogenes in SM4SP8A is determined to be 9.4×10⁶ genomes per mL. The concentration of the peptidoglycan-rich extract in SM4SP8A is determined to be 1.81 μg/mL. The concentration of the lipopolysaccharide-rich extract in SM4SP8A is determined to be 3.05 μg/mL. The concentration of the lipoteichoic-acid-rich extract in SM4SP8A is determined to be 1.85 μg/mL.

Before comparing the two cultures, the concentrations of Gram-negative DNA, Gram-positive DNA, peptidoglycan, lipopolysaccharide, and lipoteichoic acid in SM4SP8B are normalized to agree most closely with the corresponding concentrations in SM4SP8A from Example 3 (which are, respectively, 1.7×10⁸ genomes per mL, 5.8×10⁶ genomes per mL, 1.15 μg/mL, 2.08 μg/mL, and 1.22 μg/mL). The normalization factor is the amount by which each of the five concentrations in SM4SP8B must be adjusted through dilution or evaporation to obtain the culture most similar to SM4SP8A.

The normalization factor is a function of the concentrations of the two substances in SM4SP8B that deviate from the concentrations in SM4SP8A by the largest and smallest amounts. Since the concentrations in SM4SP8B differ from the concentrations in SM4SP8A by +64.7% for Gram-negative DNA, +62.1% for Gram-positive DNA, +57.4% for peptidoglycan, +46.6% for lipopolysaccharide, and +51.6% for lipoteichoic acid, the two substances used to calculate the normalization factor are Gram-negative DNA and lipopolysaccharide. In the normalized SM4SP8B culture, as compared with SM4SP8A, the percentage deviations of Gram-negative DNA and lipopolysaccharide are identical, but of different signs, and the percentage deviations of the other three substances lie somewhere in between.

The normalization factor is calculated by solving the equation:
{([DNA2]×NF)/[DNA1]}+{([LPS2]×NF)/[LPS1]}=2
wherein NF is the normalization factor; [DNA1] is the Gram-negative DNA concentration in SM4SP8A; [DNA2] is the Gram-negative DNA concentration in SM4SP8B; [LPS1] is the lipopolysaccharide concentration in SM4SP8A; and [LPS2] is the lipopolysaccharide concentration in SM4SP8B.

The equation yields a normalization factor of 0.6424, which means that normalization is accomplished by dilution. In order to reduce the concentration of each measured substance by a factor of 0.6462, the volume of the normalized culture must be equal to the reciprocal percentage of 0.6462, or 154.75% of the original volume.

In the present example, normalization of SM4SP8B by a factor of 0.6424 yields a normalized SM4SP8B culture that, as compared with SM4SP8A, contains 5.80% more Gram-negative DNA, 3.44% more Gram-positive DNA, 0.87% more peptidoglycan, 5.80% less lipopolysaccharide, and 2.45% less lipoteichoic acid. Therefore, the two cultures are equivalent because the variation in concentration of each of the three measured substances is less than 10%.

### Example 6

### Formulating a Characterized Multi Bacterial Vaccine

100 mL of SM144A is combined with 100 nL of SP288A, heat-sterilized for two hours at 65° C., lyophilized, reconstituted with 200 mL of bacteriostatic water for injection, and packaged in 1 mL sterile vials.

### Example 7

### Administering a Therapeutically-effective Amount of a Characterized Multibacterial Vaccine in the Treatment of cancer

The initial dosage is determined by titration, beginning with administration of a dose of 0.01 mL directly into a primary tumour or metastasis, or, if inaccessible, as close to a primary tumour or metastasis as possible, until the patient responds with chills followed by a minimum fever of 39° C. within two hours of injection. If there is no minimum reaction, the dose is doubled to 0.02 mL. If there is still no reaction, the dose is increased by 0.02 mL, until the minimum reaction is produced. Dosage is held constant until a minimum fever of 39° C. is no longer achieved. Dosage is then increased by increments of 0.02 mL until the minimum fever is achieved. Treatment is provided daily, until most or all of the clinically-apparent disease has regressed, followed by three times weekly for 26 weeks, and then once weekly for 52 weeks.

### Example 8

### Characterizing a Gram-positive Bacteria Broth Media Culture

A Gram-positive bacterial culture was prepared in accordance with the following method.

A 48-hour broth culture containing 25 mL of sterile (autoclaved, 20 m, 121° C., 15 atm) broth media consisting of water (JT Baker, 4216, USP certified), 10 g/L Bacto peptone (Becton Dickinson, 211677, certified USA animal origin), 3g/L beef extract (Becton Dickinson, 212303, certified USA animal origin), and 5 g/L NaCl (JT Baker, 3628-01, USP certified) was seeded with a Gram-positive bacterial seed stock (Streptococcus pyogenes NY5) and grown at 37° C. in an incubator for 48 hours. 70 mL of broth media (prepared as above) was inoculated with a 0.8-mL aliquot of the 48-hour broth culture, and grown at 37° C. in an incubator for 21 days. The resulting broth culture, designated “SP21 A”, was heat sterilized for 1 hour at 75° C.

The relative DNA concentration was determined as the product of the number of bacterial cells per mL (common log base 9) multiplied by the whole-genome size (millions of base pairs). The exact number of immunostimulatory DNA sequences within Streptococcus pyogenes NY5, or any other bacterial genome, is unknown. However, since each genome of Streptococcus pyogenes NY5 contains the identical sequences, the relative DNA concentration is directly proportional to the absolute concentration of immunostimulatory sequences.

The number of bacterial cells per mL of the broth culture was determined by directly counting the number of bacterial cells in a dilution of the broth culture using a Petroff Hausser Counting Chamber (Hausser Scientific, 3900). The counting chamber had 0.05 mm square fields, 0.02 mm in depth, containing a volume of 5×10⁻⁵ mm³ or 5×10⁻⁸ mL. The bacteria in sixteen fields were counted at 720-power magnification, and the results were averaged and adjusted by the dilution factor. The bacterial cell concentration of SP21A was 58×10⁹ cells/mL; the Streptococcus pyogenes genome consists of approximately 1.85 Mb (Ferretti, 2001); and the relative DNA concentration of SP21A was determined to be 107 (1.85×58).

The relative concentration of peptidoglycan was determined by a colorimetric assay for lactic acid (Barker and Summerson, 1940), adapted for the quantitative determination of muramic acid in bacterial peptidoglycan (Hadzija, 1974). Peptidoglycan exists as a complex polymer, but each subunit of peptidoglycan contains one copy of muramic acid; therefore, the relative concentration of peptidoglycan is directly proportional to the absolute concentration of muramic acid.

Bacteria were harvested from 15 mL of SP21 A broth culture by centrifugation (60 min, 1,300×g), washed three times in 10 mL of deionized ultra-filtered water (Fisher Scientific, W2-4), and resuspended in 2 mL of deionized ultra-filtered water in a test tube. To break the cell walls, 1.0 g of glass beads (diameter 0.106 mm, Sigma, G8893) were added, and the test tube was vortexed on high power (Dade Multi-Tube vortexer) for 30 min. After a 15-min settling time, 0.4 mL of the supernatant was transferred to a 16 mm×100 mm pyrex test tube with a teflon-lined screw cap; 0.2 mL (2 N) NaOH (Fisher Scientific, SS255B, 1:4 dilution) was added, and the tube was capped, shaken, and allowed to incubate in a water bath for 30 min at 38° C.

To the sample was added 0.4 mL (N) of H₂SO₄ (Fisher, SA212B), then 0.05 mL of CuSO₄ (4% w/v, Sigma, C2284), then 5.0 mL of concentrated H₂SO₄ (Fisher, A300). The tube was tightly capped, vortexed (VWR Mini-vortexer) on high for 2 sec, placed in a boiling water bath for 7 min, cooled under running tap water, and then allowed to incubate in an ice water bath (10° C.) for 5 min. 0.1 mL of 1.5% (w/v) p-hydroxybiphenyl (ICN Biomedicals, 141290) was then added to the sample. The contents were thoroughly mixed by shaking and vortexing, and then placed in a shaker water bath (Precision, model 51221079) at 29.9° C. and 50 rpm for 35 min. During this incubation period, the tube was removed from the bath and briefly shaken at 6, 12, 18, 25, and 31 min. The sample was placed in a 98° C. water bath for 75 sec, cooled under running tap water, and then allowed to incubate in an ice water bath (10° C.) for 5 min. The sample was read on a spectrophotometer (Milton Roy, model 301) at 560 nm against a reagent blank. The measured absorbance of .016 for the 0.4-mL broth sample corresponds to 2.5 μg of muramic acid per mL; therefore, the relative concentration of peptidoglycan in the sample was 2.5.

### Example 9

### Characterizing a Gram-negative Bacteria Agar Media Culture

A Gram-negative bacterial culture was prepared in accordance with the following method.

A 48-hour broth culture containing 25 mL of sterile (autoclaved, 20m, 121° C., 15 atm) broth media consisting of water (JT Baker, 4216, USP certified), 10 g/L Bacto peptone (Becton Dickinson, 211677, certified USA animal origin), 3g/L beef extract (Becton Dickinson, 212303, certified USA animal origin), and 5 g/L NaCl (JT Baker, 3628-01, USP certified) was seeded with a Gram-negative bacterial seed stock (Serratia marcescens Bizio) and grown at 25° C. in an incubator for 48 hours. 2% (w/v) agar (Sigma, A6686) was dissolved in 360 mL of broth media (prepared as above), autoclaved (20 m, 121° C., 15 atm), and then poured to a depth of 1 cm in six sterile 15 mm×100 mm petri dishes. After the agar media had set, the petri dishes were inoculated with 0.8-mL aliquots of the 48-hour broth culture and grown at 21° C. in an incubator for 7 days. The bacteria were harvested from the agar surface, heat sterilized (75° C., 1 h), centrifuged (30 min, 1,300×g), and separated into wet bacteria and supernatant fractions. The wet bacteria were rubbed up into a thick suspension using a mortar and pestle; this was then washed into a test tube with a small quantity of the supernatant.

The suspension and the supernatant were analyzed for organic nitrogen by the Kjeldahl method, which involves converting the organic nitrogen into ammonia ion by acidic digestion, converting the ammonia ion into ammonia gas by alkaline hydrolysis, collecting the ammonia gas by distillation, and then measuring the amount of nitrogen by titration. Briefly, 0.5-mL aliquots of the bacterial suspension and of the supernatant were added to 3 mL of deionized ultra-filtered water (Fisher Scientific, W2-4), 2 g of K₂SO₄ (Fisher Scientific, P304), 0.094 g of CuSO₄.5H₂O (Sigma, C7631), and 5 mL of concentrated H₂SO₄ (Fisher Scientific, A300) in a 1000-mL digestion flask. The mixture was refluxed at approximately 400° C. for 42 min, and then allowed to cool to room temperature. The digestate was diluted with 160 mL of deionized ultra-filtered water. 25 mL (10N) of NaOH (Fisher Scientific, SS255B) were then poured down the side of the flask to form a dense layer beneath the diluted digestate. The flask was connected to a distillation apparatus, and then swirled to mix. Full heat was applied, and the distillate was collected in a flask containing 50 mL of a 2:1 dilution of Kjel-Sorb (Fisher Scientific, SK-15) in deionized ultra-filtered water. After 100 mL of distillate were collected, the distillate was titrated with .02 N H₂SO₄ (Fisher Scientific, SA226B) to a bright red endpoint. The bacterial suspension distillate reached its endpoint on addition of 6.5 mL (0.02N) of acid; the suspension contained 0.135 moles per 0.5 mL of sample or 3.64 mg of nitrogen per mL. The supernatant contained 0.357 mg of nitrogen per mL. The supernatant and the suspension were mixed in equal volumes. The resulting culture suspension, designated “SM7A”, contained 2.0 mg of nitrogen per mL.

The relative DNA concentration was determined as the product of the number of bacterial cells per mL (common log base 9) multiplied by the whole-genome size (millions of base pairs). The exact number of immunostimulatory DNA sequences within Serratia marcescens Bizio, or any other bacterial genome, is unknown; however, since each genome of Serratia marcescens Bizio contains the identical sequences, the relative DNA concentration is directly proportional to the absolute concentration of immunostimulatory sequences.

The number of bacterial cells per mL of the culture suspension was determined by directly counting the number of bacterial cells in a dilution of the culture suspension using a Petroff Hausser Counting Chamber (Hausser Scientific, 3900). The counting chamber had 0.05 mm square fields, 0.02 mm in depth, containing a volume of 5×10⁻⁵ mm³ or 5×10⁻⁸ mL. The bacteria in sixteen fields were counted at 720-power magnification, and the results were averaged and adjusted by the dilution factor. The bacterial cell concentration of SM7A was 29×10⁹ cells/mL; the Serratia marcescens genome consists of approximately 5.11 Mb (Sanger Institute, 2006); and the relative DNA concentration of SM7A was determined to be 148 (5.11×29).

The relative concentration of peptidoglycan was determined by a colorimetric assay for lactic acid (Barker and Summerson, 1941), adapted for the quantitative determination of muramic acid in bacterial peptidoglycan (Hadzija, 1974). Peptidoglycan exists as a complex polymer. Since each subunit of peptidoglycan contains one copy of muramic acid, the relative concentration of peptidoglycan is directly proportional to the absolute concentration of muramic acid.

1 mL of SM7A culture suspension was diluted with 9 mL of deionized ultra-filtered water (Fisher Scientific, W2-4), centrifuged (60 min, 1,300×g), washed two times in 10 mL of deionized ultra-filtered water, and resuspended in 2 mL of deionized ultra-filtered water in a test tube. To break the cell walls, 1.0 g of glass beads (diameter 0.106 mm, Sigma, G8893) was added, and the test tube was vortexed on high power (Dade Multi-Tube vortexer) for 30 min. After a 15-min settling time, 0.4 mL of the supernatant was transferred to a 16 mm×100 mm pyrex test tube with a teflon-lined screw cap. 0.2 mL (2 N) of NaOH (Fisher Scientific, SS255B, 1:4 dilution) was added, and the tube was capped, shaken, and allowed to incubate in a water bath for 30 min at 38° C.

To the sample were added 0.4 mL (N) of H₂SO₄ (Fisher, SA212B), then 0.05 mL of CuSO₄ (4% w/v, Sigma, C2284), then 5.0 mL of concentrated H₂SO₄ (Fisher, A300). The tube was tightly capped, vortexed (VWR Mini-vortexer) on high for 2 sec, placed in a boiling water bath for 7 min, cooled under running tap water, and allowed to incubate in an ice water bath (10° C.) for 5 min. To the sample was added 0.1 mL of 1.5% (w/v) p-hydroxybiphenyl (ICN Biomedicals, 141290). The contents were thoroughly mixed by shaking and vortexing, and then placed in a shaker water bath (Precision, model 51221079) at 29.9° C. and 50 rpm for 35 min. During this incubation period, the tube was removed from the bath and briefly shaken at 6, 12, 18, 25, and 31 min. The sample was placed in a 98° C. water bath for 75 sec, cooled under running tap water, and then allowed to incubate in an ice water bath (10° C.) for 5 min. The sample was read on a spectrophotometer (Milton Roy, model 301) at 560 nm against a reagent blank. The observed absorbance of .217 for the 0.4-mL suspension sample corresponds to 33.9 μg of muramic acid per mL; therefore, the relative concentration of peptidoglycan in the sample was 33.9.

### Example 10

### Formulating a Characterized Mixed Bacterial Vaccine (Coley Vaccine)

Coley vaccines are a class of mixed bacterial vaccines comprised of Gram-positive bacteria species Streptococcus pyogenes and Gram-negative bacteria species Serratia marcescens. Coley Vaccine Type XI, the formulation used in the largest number of successful cases (Nauts, 1953), was prepared in the following ratio: 100 parts Streptococcus pyogenes broth culture; 30 parts Serratia marcescens culture suspension with 2 mg of nitrogen per mL; 20 parts glycerine; and 0.5% (w/v) thymol as a preservative (Coley, 1909).

The characterized Coley Vaccine MBV1 was formulated by combining 76.7 mL of broth culture SP21A, 23 mL of culture suspension SM7A, 15.3 mL of glycerin (JT Baker, 2142, USP certified), and 0.5% (w/v) thymol (JT Baker, 4128, NF certified). The relative concentrations (relative concentration per mL of each culture multiplied by volume of each culture divided by total volume of the vaccine) of the four immunostimulatory substances in MBV1 were: 29.6 for Gram-negative bacterial DNA, 71.3 for Gram-positive bacterial DNA, 6.8 for Gram-negative peptidoglycan, and 1.7 for Gram-positive peptidoglycan.

### Example 11

### Reproducing a Previously-characterized Mixed Bacterial Vaccine (Coley Vaccine)

Because bacterial growth cannot be exactly controlled, batches of the same bacteria, grown under the same conditions, can have differing concentrations of bacteria and bacterial immunostimulatory substances.

The characterized Coley Vaccine MBV1 was formulated by combining Streptococcus pyogenes broth culture SP21A (with relative DNA and peptidoglycan concentrations of 107 and 2.5 per mL, respectively) and Serratia marcescens culture suspension SM7A (with relative DNA and peptidoglycan concentrations of 148 and 33.9 per mL, respectively). The relative DNA and peptidoglycan concentrations of a new Streptococcus pyogenes broth culture SP21B were 120 and 2.7 per mL, respectively. The relative DNA and peptidoglycan concentrations of a new Serratia marcescens culture suspension SM7B were 107 and 26.8 per mL, respectively.

SP21B is more concentrated than SP21A, and SM7B is less concentrated than SM7A. Therefore, to formulate MBV2 from cultures SP21 B and SM7B, such that it most closely reproduces the immunostimulatory component ratios of MBV1, broth culture SP21B must be diluted. The amount of dilution is calculated by averaging the DNA and peptidoglycan ratios of the two sets of cultures, as described below.

For example, where the DNA ratio (100 mL of SP21A: 30 mL of SM7A) is 2.41, and the DNA ratio (100 mL of SP21B: 30 mL of SM7B) is 3.74, the DNA ratios are equalized by diluting SP21B by a factor of 0.644 (2.41/3.74). Where the peptidoglycan ratio (100 mL of SP21A: 30 mL of SM7A) is 0.25, and the peptidoglycan ratio (100 mL of SP21B :30 mL of SM7B) is 0.34, the peptidoglycan ratios are equalized by diluting SP21B by a factor of 0.735 (0.25/0.34). The average dilution factor is 0.690 ((0.644+0.735)/2). Therefore, 69 mL of the Streptococcus pyogenes broth culture SP21B is diluted to a final volume of 100 mL with USP water. The relative DNA and peptidoglycan concentrations of the diluted Streptococcus pyogenes broth culture SP21B2 are 82.8 and 1.86 per mL, respectively.

In accordance with the foregoing calculation, the DNA ratio of MBV2 (100 mL of SP21B2: 30 mL of SM7B) is 2.58-7% higher than the DNA ratio of MBV1, which is 2.42 (100 mL of SP21A: 30 mL of SM7A). Additionally, the peptidoglycan ratio of MBV2 (100 mL of SP21B2 :30 mL of SM7B) is 0.23-8% lower than the peptidoglycan ratio of MBV1, which is 0.25 (100 mL of SP21A: 30 mL of SM7A).

### REFERENCES CITED

1. Barker and Summerson, Colorimetric determination of lactic acid in biological material, 1940.
2. Bauer et al., Human TLR9 confers responsiveness to bacterial DNA via species-specific CpG motif recognition. PNAS, 98(16):9237, 2001.
3. Beachey et al., Lymphocyte binding and T cell mitogenic properties of group A streptococcal lipoteichoic acid. J. Immunol., 122:189, 1979.
4. Beers and Berkow (eds.), The Merck Manual of Diagnosis and Therapy, 17^{th} ed. (Whitehouse Station, N.J.: Merck Research Laboratories, 1999) 973-74, 976, 986, 988, 991.
5. Bhakdi and Tranum-Jensen, Complement activations and attack on autologous cell membranes induced by streptolysin O. Inf. Immunity, 48(3):713, 1985.
6. Bhakdi et al., Stimulation of monokine production by lipoteichoic acids. Infect. Immun., 59:4614, 1991.
7. Bjork et al., Endotoxin and Staphylococcus aureus enterotoxin A induce different patterns of cytokines. Cytokine, 4:513, 1992.
8. Cleveland et al., Lipoteichoic acid preparations of gram-positive bacteria induce interleukin-12 through a CD14-dependent pathway. Inf. Immunity, 64(6):1906, 1996.
9. Coley, W. B., Late results of the treatment of inoperable sarcoma by the mixed toxins of erysipelas and Bacillus prodigiosus. Am. J. Med. Sci., 131:375-430, 1906.
10. Coley, W. B., The treatment of inoperable sarcoma by bacterial toxins (the mixed toxins of the streptococcus of erysipelas and the Bacillus prodigiosus), 596-97, November 1909.
11. Coley Pharmaceuticals, www.coleypharma.com, accessed Dec. 17, 2003.
12. Cunningham et al., Further purification of a group A streptococcal pyrogenic exotoxin and characterization of the purified toxin. Inf. Immunity, 14(3):767, 1976.
13. de Jonge et al., Peptidoglycan composition of a highly methicillin-resistant Staphylococcus aureus strain. J. Biol. Chem., 27(16):11248, 1992.
14. Dziarski, R., Studies on the mechanism of peptidoglycan- and lipopolysaccharide-induced polyclonal activation. Inf. Immunity, 35(2):507, 1982.
15. Dziarski et al., Binding of bacterial peptidoglycan to CD14. J. Biol. Chem., 273:8680, 1998.
16. Farias-Eisner et al., Nitric oxide is an important mediator for tumoricidal activity in vivo. PNAS, 91:9407, 1994.
17. Fast et al., Toxic shock syndrome-associated staphylococcal and streptococcal pyrogenic toxins are potent inducers of tumor necrosis factor production. Infect. Immunity, 57:291, 1989.
18. Ferretti et al., Complete genome sequence of an M1 strain of Streptococcus pyogenes. PNAS, 98(8):4658, 2001.
19. Fischer et al., Improved preparation of lipoteichoic acids. Eur. J. Biochem., 133:523, 1983.
20. Galanos et al., Preparation and properties of a standard lipopolysaccharide from Salmonella abortus equi (Novo-Pyrexal). Zentral Bakt. Mikrobiol. Hyg. I Abt Orig. A, 243:226, 1979.
21. Gao et al., Commercial preparation of lipoteichoic acid contain endotoxin that contributes to activation of mouse macrophages in vitro. Inf. Immunity, 69(2):751, 2001.
22. Hackett and Stevens, Streptococcal toxic shock syndrome: synthesis of tumor necrosis factor and interleukin-1 by monocytes stimulated with pyrogenic exotoxin A and streptolysin O. J. Infect. Dis., 165:879, 1992.
23. Hackett and Stevens, Superantigens associated with staphylococcal and streptococcal toxic shock syndrome are potent inducers of tumor necrosis factor-beta synthesis. J. Infect. Dis., 168:232, 1993.
24. Hadzija, O., A simple method for the qualitative determination of muramic acid. Analytical Biochem., 60:512-17, 1974.
25. Hartmann et al., CpG DNA: a potent signal for growth, activation, and maturation of human dendritic cells. PNAS, 96:9305, 1999.
26. Havas et al., Mixed bacterial toxins in the treatment of tumors. I. Methods of preparation and effects on normal and sarcoma 37-bearing mice. Cancer Res., 18:141, 1958.
27. Hoption Cann et al., Tumor regression: a place in history or in the future. Postgrad. Med. J., 79:672, 2003.
28. Houston and Ferretti, Enzyme-linked immunosorbant assay for detection of type A Streptococcal exotoxin: kinetics and regulation during growth of Streptococcus pyogenes. Inf. and Immunity, 33(3):862, 1981.
29. Itoh et al., Mechanism of stimulation of T cells by Streptococcus pyogenes: isolation of a major mitogenic factor, cytoplasmic membrane-associated protein. Inf. and Immunity, 60(8):3128, 1992.
30. Keller et al., Macrophage response to bacteria: induction of marked secretory and cellular activities by lipoteichoic acids. Infect. Immunity, 60:3664, 1992.
31. Leonard et al., Cell and receptor requirements for streptococcal pyrogenic exotoxin T cell mitogenicity. Infect. Immunity, 59:1210, 1991.
32. Leulier et al., The Drosophila immune system detects bacteria through specific peptidoglycan recognition. Nature Immunol., 4:478, 2003.
33. Loos et al., Interaction of purified lipoteichoic acid with the classical complement pathway. Inf. Immunity, 53(3):595, 1986.
34. Luchi and Morrison, Comparable endotoxic properties of lipopolysaccharides are manifest in diverse clinical isolates of gram-negative bacteria. Infect. Immunity, 68(4):1899, 2000.
35. Luster and Leder, IP-10, a CXC chemokine, elicits a potent thymus-dependent antitumor response in vivo. J. Exp. Med., 178:1057-65, 1996.
36. Marrack and Kappler, The staphylococcal enterotoxins and their relatives. Science, 248:705, 1990.
37. Matsuura et al., Activity of monosaccharide lipid A analogues in human monocytic cells as agonists or antagonists of bacterial lipopolysaccharide. Inf. and Immunity, 67(12):6286, 1999.
38. Matzinger, P., An innate sense of danger. Sem. Immunology, 10:399, 1998.
39. Muller-Alouf et al., Pyrogenicity and cytokine-inducing properties of Streptococcus pyogenes superantigens: comparative study of streptococcal mitogenic exotoxin Z and pyrogenic exotoxin A. Inf. and Immunity, 69(6):4141, 2001.
40. Nauts et al., A review of the influence of bacterial infection and of bacterial products (Coley's toxins) on malignant tumors in man. Acta Med. Scand. Suppl., 276:1-103; and Appendix, “Index of various preparations of Coley's Toxins used”, 1953.
41. Nauts, H. C., Beneficial effects of immunotherapy (bacterial toxins) on sarcoma of the soft tissues, other than lymphosarcoma. End results in 186 determinate cases with microscopic confirmation of diagnosis—49 operable, 137 inoperable. Cancer Research Inst., Monograph #16, 1975.
42. Nauts, H. C., Bacterial pyrogens: beneficial effects on cancer patients. Prog. Clin. Biol. Res., 107:687-96, 1982.
43. Nauts, H. C., Breast cancer: Immunological factors affecting incidence, prognosis and survival. Cancer Research Inst., Monograph #18, 1984.
44. Nauts and McLaren, Coley toxins—the first century. Adv. Exp. Med. Biol., 267:483-500, 1990.
45. Spratto and Woods (eds.), PDR Nurses Drug Handbook (Montvale, N.J.: Delmar, 2002).
46. Richardson et al., Coley toxins immunotherapy: a retrospective review. Altern. Ther. Health Med., 5(3):42-47, 1999.
47. Sakamoto et al., Meta-analysis of adjuvant immunochemotherapy using OK-432 in patients with resected non-small-cell lung cancer. J. Immunotherapy, 24(3):250, 2001.
48. Sanger Institute, Complete genome sequence of Serratia marcescens—www.sanger.ac.uk/Projects/S_marcescens/
49. Schwandner et al., Peptidoglycan- and lipoteichoic acid-induced cell activation is mediated by toll-like receptor 2. J. Biol. Chem., 274(25):17406,1999.
50. SEER Cancer Statistics Review, 1973-1999, Table 1-3. National Cancer Institute website www.seer.cancer.gov, accessed Apr. 2, 2003.
51. Stassen et al., The streptococcal exotoxin streptolysin O activates mast cells to produce tumor necrosis factor alpha by p38 mitogen-activated protein kinase- and protein kinase C-dependent pathways. Inf. Immunity, 71(11):6171, 2003.
52. Tsutsui et al., Relationship of the chemical structure and immunobiological activities of lipoteichoic acid from Streptococcus faecalis (Enterococcus hirae) ATCC 9790. FEMS Microbiol. Immunol., 76:211, 1991.
53. Usami et al., Antitumor effects of streptococcal lipoteichoic acids on Meth A fibrosarcoma. Br. J. Cancer, 57:70, 1988.
54. Waisbren, B. A., Observations on the combined systemic administration of mixed bacterial vaccine, bacillus Calmette-Guerin, transfer factor, and lymphoblastoid lymphocytes to patients with cancer, 1974-1985. J. Biol. Response Mod., 6(1):1-19, 1987.
55. Wang et al., Peptidoglycan and lipoteichoic acid from Staphylococcus aureus induce tumor necrosis factor alpha, interleukin 6 (IL-6), and IL-10 production in both T cells and monocytes in a human whole blood model. Inf. and Immunity, 68(7):3965, 2000.
56. Wang et al., Micrococci and peptidoglycan activate TLR2>MyD88>IRAK>TRAF>NIK>IKK>NF-kappa-B signal transduction pathway that induces transcription of interleukin-8. Inf. Immunity, 69(4):2270, 2001.
57. Weidemann et al., Soluble peptidoglycan-induced monokine production can be blocked by anti-CD14 monoclonal antibodies and by lipid A parital structures. Inf. Immunity, 62(11):4709, 1994.
58. Wiemann and Stames, Coley's toxins, tumor necrosis factor and cancer research: a historical perspective. Pharmacol. Ther., 64(3):529-64, 1994.
59. Yamamoto et al., The use of lipoteichoic acid (LTA) from Streptococcus pyogenes to induce a serum factor causing tumor necrosis. Br. J. Cancer, 51:739, 1985.
60. Zhang et al., Streptococcal histone induces murine macrophages to product interleukin-1 and tumor necrosis factor alpha. Inf. Immunity, 67(12):6473, 1999.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for establishing a standard for a Gram-negative bacterial culture, comprising determining the relative concentrations of at least two immunostimulatory bacterial substances in the culture.

2. The method of claim 1, wherein the at least two immunostimulatory bacterial substances are selected from the group consisting of bacterial DNA, peptidoglycan, and lipopolysaccharide.

3. The method of claim 1, wherein the relative concentrations of bacterial DNA, peptidoglycan, and lipopolysaccharide in the culture are determined.

4. The method of claim 1, wherein the Gram-negative bacterial culture comprises Serratia marcescens.

5. A method for establishing a standard for a Gram-positive bacterial culture, comprising determining the relative concentrations of at least two immunostimulatory bacterial substances in the culture.

6. The method of claim 5, wherein the at least two immunostimulatory bacterial substances are selected from the group consisting of bacterial DNA, peptidoglycan, and lipoteichoic acid.

7. The method of claim 5, wherein the relative concentrations of bacterial DNA, peptidoglycan, and lipoteichoic acid in the culture are determined.

8. The method of claim 5, wherein the Gram-positive bacterial culture comprises Streptococcus pyogenes.

9. A method for establishing a standard for a mixed bacterial culture comprising at least one Gram-negative bacterium and at least one Gram-positive bacterium, the method comprising determining the relative concentrations of at least two immunostimulatory bacterial substances in the mixed bacterial culture.

10. The method of claim 9, wherein the at least two immunostimulatory bacterial substances are selected from the group consisting of Gram-negative bacterial DNA, Gram-positive bacterial DNA, peptidoglycan, lipopolysaccharide, and lipoteichoic acid.

11. The method of claim 9, wherein the relative concentrations of Gram-negative bacterial DNA, Gram-positive bacterial DNA, peptidoglycan, lipopolysaccharide, and lipoteichoic acid in the culture are determined.

12. The method of claim 9, wherein the mixed bacterial culture comprises a Coley vaccine.

13. A method for reproducing a Gram-negative bacterial culture, comprising the steps of:
(a) obtaining a first Gram-negative bacterial culture;
(b) determining the relative concentrations of at least two immunostimulatory bacterial substances in the first culture;
(c) obtaining a second Gram-negative bacterial culture;
(d) determining the relative concentrations of the at least two immunostimulatory bacterial substances in the second culture; and
(e) normalizing the second Gram-negative bacterial culture.

14. The method of claim 13, wherein the at least two immunostimulatory bacterial substances are selected from the group consisting of bacterial DNA, peptidoglycan, and lipopolysaccharide.

15. The method of claim 13, wherein the relative concentrations of bacterial DNA, peptidoglycan, and lipopolysaccharide in the first culture and in the second culture are determined.

16. The method of claim 13, further comprising the step of determining the degree of equivalence between the normalized second culture and the first culture.

17. A method for reproducing a Gram-positive bacterial culture, comprising the steps of:
(a) obtaining a first Gram-positive bacterial culture;
(b) determining the relative concentrations of at least two immunostimulatory bacterial substances in the first culture;
(c) obtaining a second Gram-positive bacterial culture;
(d) determining the relative concentrations of the at least two immunostimulatory bacterial substances in the second culture; and
(e) normalizing the second Gram-positive bacterial culture.

18. The method of claim 17, wherein the at least two immunostimulatory bacterial substances are selected from the group consisting of bacterial DNA, peptidoglycan, and lipoteichoic acid.

19. The method of claim 17, wherein the relative concentrations of bacterial DNA, peptidoglycan, and lipoteichoic acid in the first culture and in the second culture are determined.

20. The method of claim 17, further comprising the step of determining the degree of equivalence between the normalized second culture and the first culture.

21. A method for reproducing a mixed bacterial culture comprising at least one Gram-negative bacterium and at least one Gram-positive bacterium, the method comprising the steps of:
(a) obtaining a first mixed bacterial culture;
(b) determining the relative concentrations of at least two immunostimulatory bacterial substances in the first culture;
(c) obtaining a second mixed bacterial culture;
(d) determining the relative concentrations of the at least two immunostimulatory bacterial substances in the second culture; and
(e) normalizing the second mixed bacterial culture.

22. The method of claim 21, wherein the at least two immunostimulatory bacterial substances are selected from the group consisting of Gram-negative bacterial DNA, Gram-positive bacterial DNA, peptidoglycan, lipopolysaccharide, and lipoteichoic acid.

23. The method of claim 21, wherein the relative concentrations of Gram-negative bacterial DNA, Gram-positive bacterial DNA, peptidoglycan, lipopolysaccharide, and lipoteichoic acid in the first culture and in the second culture are determined.

24. The method of claim 21, further comprising the step of determining the degree of equivalence between the normalized second culture and the first culture.

25. A method for preparing a multibacterial vaccine, comprising the steps of:
(a) obtaining a Gram-negative bacterial culture;
(b) determining the relative concentrations of bacterial DNA, peptidoglycan, and lipopolysaccharide in the Gram-negative bacterial culture;
(c) obtaining a Gram-positive bacterial culture;
(d) determining the relative concentrations of bacterial DNA, peptidoglycan, and lipoteichoic acid in the Gram-positive bacterial culture; and
(e) combining the Gram-negative bacterial culture and the Gram-positive bacterial culture.

26. A multibacterial vaccine prepared in accordance with the method of claim 25.

27. A method for treating and/or preventing a disorder in a subject, comprising administering to the subject the multibacterial vaccine of claim 26, in an amount effective to treat and/or prevent the disorder in the subject.

28. The method of claim 27, wherein the disorder is selected from the group consisting of a bum, an infection, neoplasia, and a radiation injury.

29. A method for preparing a multibacterial vaccine, comprising the steps of:
(a) obtaining a mixed bacterial culture comprising a Gram-negative bacterial culture and a Gram-positive bacterial culture; and
(b) determining the relative concentrations of Gram-negative bacterial DNA, Gram-positive bacterial DNA, peptidoglycan, lipopolysaccharide, and lipoteichoic acid in the mixed bacterial culture.

30. A multibacterial vaccine prepared in accordance with the method of claim 29.

31. A method for treating and/or preventing a disorder in a subject, comprising administering to the subject the multibacterial vaccine of claim 30, in an amount effective to treat and/or prevent the disorder in the subject.

32. The method of claim 31, wherein the disorder is selected from the group consisting of a bum, an infection, neoplasia, and a radiation injury.

33. A method for predicting the efficacy of a multibacterial vaccine, comprising the steps of:
(a) obtaining a first multibacterial vaccine having efficacy in the treatment and/or prevention of at least one disorder;
(b) determining the relative concentrations of at least two immunostimulatory bacterial substances in the first multibacterial vaccine;
(c) obtaining a second multibacterial vaccine;
(d) determining the relative concentrations of the at least two immunostimulatory bacterial substances in the second multibacterial vaccine; and
(e) comparing the relative concentrations in the second multibacterial vaccine with the relative concentrations in the first multibacterial vaccine, wherein the second multibacterial vaccine is more efficacious if the relative concentrations in the second multibacterial vaccine are more similar to the relative concentrations in the first multibacterial vaccine, and wherein the second multibacterial vaccine is less efficacious if the relative concentrations in the second multibacterial vaccine are less similar to the relative concentrations in the first multibacterial vaccine.

34. The method of claim 33, wherein the at least two immunostimulatory bacterial substances are selected from the group consisting of Gram-negative bacterial DNA, Gram-positive bacterial DNA, peptidoglycan, lipopolysaccharide, and lipoteichoic acid.

35. The method of claim 33, wherein the relative concentrations of Gram-negative bacterial DNA, Gram-positive bacterial DNA, peptidoglycan, lipopolysaccharide, and lipoteichoic acid in the first vaccine and in the second vaccine are determined.

36. The method of claim 33, wherein the first multibacterial vaccine is a Coley vaccine.

37. The method of claim 33, wherein the disorder is selected from the group consisting of a burn, an infection, neoplasia, and a radiation injury.

38. A method for enhancing the efficacy of a multibacterial vaccine, comprising the steps of:
(a) obtaining a first multibacterial vaccine having efficacy in the treatment and/or prevention of at least one disorder;
(b) determining the relative concentrations of at least two immunostimulatory bacterial substances in the first multibacterial vaccine;
(c) obtaining a second multibacterial vaccine;
(d) determining the relative concentrations of the at least two immunostimulatory bacterial substances in the second culture; and
(e) normalizing the second multibacterial vaccine.

39. The method of claim 38, wherein the at least two immunostimulatory bacterial substances are selected from the group consisting of Gram-negative bacterial DNA, Gram-positive bacterial DNA, peptidoglycan, lipopolysaccharide, and lipoteichoic acid.

40. The method of claim 38, wherein the relative concentrations of Gram-negative bacterial DNA, Gram-positive bacterial DNA, peptidoglycan, lipopolysaccharide, and lipoteichoic acid in the first vaccine and in the second vaccine are determined.

41. The method of claim 38, wherein the first multibacterial vaccine is a Coley vaccine.

42. The method of claim 38, wherein the disorder is selected from the group consisting of a bum, an infection, neoplasia, and a radiation injury.
